(19) United States
(12) Patent Application Publication
(10) Pub. No.: US 2002/0035350 A1
(43) Pub. Date: Mar. 21, 2002

(54) Single use syringe

(76) Inventors: Andrew W. Turnbull, Edmonton (CA); Devin C. Swanson, Hughenden (CA); Sven Gustav J. Eriksson, Stockholm (SE)

Correspondence Address: EDWARD YOO C/O BENNETT JONES, 1000 ATCO CENTRE, 10035 - 105 STREET, EDMONTON, ALBERTA, AB, T5J3T2, CA

(21) Appl. No.: 09/800,652

(22) Filed: Mar. 8, 2001

(30) Foreign Application Priority Data
Sep. 18, 2000 (CA) 2,320,245

(51) Int. Cl. A61M 5/00

(52) U.S. Cl. 604/110

(57) ABSTRACT

A non-reusable syringe utilizes a liquid-tight seal between a sliding piston assembly within an elongated barrel. The piston assembly comprises a valve assembly comprising a forward valve and a rear valve that separate upon depression of the plunger for expulsion of the liquid contents. The rear valve that a seal with the barrel and the plunger for drawing in of liquid and the forward valve that forms a seal with the barrel and the plunger for expulsion of the liquid. During liquid expulsion, the two valves separate. A bypass is provided on a plug attached to the plunger which prevents a seal from forming without the rear valve being in position. Thus, when the plunger is retracted after the first use, no liquid may be drawn up into the barrel. The syringe may further include a safety barb and closed ends to prevent tampering or manipulation of the piston apparatus in an effort to eliminate the separation of the two valves.

### FIELD OF THE INVENTION

The present invention relates to syringes and more particularly to single use disposable syringes.

### BACKGROUND OF THE INVENTION

Disposable syringes are widely used to administer substances intravenously. While it is intended that the syringes will be used once and then disposed of in an appropriate manner, problems have arisen with the repeated re-use of these disposable syringes. This phenomenon is particularly prevalent amongst intravenous drug addicts who not only repeatedly use the same syringe, but also often share it with other drug addicts. Syringe re-use is also a prevalent problem in areas where economic hardship and scarce medical resources often result in the reuse of syringes. The re-use of such disposable syringes exposes subsequent users to the risk of contracting any number of blood borne diseases including amongst others, AIDS, hepatitis and various venereal diseases. In particular, the re-use of syringes by drug addicts is believed to be a major factor in the spread of the AIDS epidemic.

Recognizing the problem with the re-use of disposable syringes, several prior patents have addressed the problem by disclosing self-destructing or single-use syringes. U.S. Pat. Nos. 4,699,614 to Glazier, 4,883,466 to Glazier, 4,908,020 to Pettersen, 5,066,280 to Braithwaite, 6,013,056 to Pettersen and French Patent 2,676,928 to Faure all utilize mechanisms whereby the plunger shaft dislocates from the piston following liquid intake and subsequent expulsion or whereby the plunger shaft is compromised following liquid intake and subsequent expulsion.

Unfortunately, the devices disclosed in these prior art patents are complex, making manufacturing costs disproportionately high. Further, many of the prior art devices are vulnerable to tampering and mechanical manipulation either before or after first use, which permits the syringe to be re-used. For example, the piston and plunger assembly can be removed and reassembled using glue or an alternate fixative in many of these prior art inventions.

Therefore, there is a need in the art for a single use syringe, which is simple and cost effective to manufacture, yet which is difficult to be mechanically manipulated for re-use.

### SUMMARY OF THE INVENTION

The present invention is directed to a single-use disposable syringe.

Accordingly, in one aspect of the invention, the invention comprises an apparatus comprising:

(a) an elongate barrel having an interior surface, a rear end and a forward restricted end having an opening through which liquid can pass, said forward end having a cone for attaching a needle;

(b) a piston disposed within the barrel comprising a first forward valve and a separate second rear valve, said first valve and second valve each forming a substantially liquid-tight seal with the interior surface of the barrel, said first valve defining a cavity and a front opening and having a front valve seat and a rear shoulder, and said second valve defining a cavity and rear opening and having a rear valve seat;

(c) an elongate plunger comprising a shaft terminating with a plug, said plug having a front sealing surface, a shoulder, a rear sealing surface and defining a bypass having an opening through the front sealing surface and another opening rearward of the shoulder;

(d) said plug disposed within the first valve cavity and the second valve cavity with the shaft passing through the rear opening of the second valve, and said valve sized and shaped such that when the plug is pulled back, the front sealing surface disengages the front valve seat, the plug shoulder engages the piston cavity shoulder, the rear sealing surface engages the rear valve seat and the plug and first and second piston valves are pulled rearward within the barrel as one unit; and when said plug is urged in a forward direction, the forward sealing surface engages the forward valve seat and the first piston valve and plug disengage the second piston valve.

In one preferred embodiment, the rear end is closed with an opening through which the plunger may pass, thereby preventing removal of the plunger-piston assembly from the barrel without destroying the barrel.

In one embodiment, the shaft comprises blocking means allowing passage of the shaft through the second piston valve in the forward direction but preventing passage of the shaft through the second piston valve in the rearward direction. The blocking means may comprise a barb fixed onto the shaft which only permits one-way movement of the rear valve over the shaft. In one embodiment, the front valve may further comprise a projection that fits into a reciprocating cavity in the second valve wherein the projection and cavity are aligned during plunger retraction and liquid intake but unaligned following plunger compression and liquid expulsion.

In another aspect, the invention comprises a single use syringe comprising:

(a) a barrel having a closed forward end and a closed rear end, said forward end defining a cone and an opening for attaching a needle;

(b) a piston assembly including a front valve and a rear valve;

(c) a plunger including a shaft which passes through the rear valve and a plug wherein said plug mates in a fluid-tight manner with the front valve and with the rear valve and defines an internal bypass opening operative when the front valve and rear valve are separated;

(d) means for coupling the plug to the front valve such that when the plug is retracted, the front valve also retracts; and

(e) wherein the plug does not engage the rear valve when urged forward.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is an elevated side view, in section, of an embodiment of the apparatus.

FIG. 2 is a schematic view of an embodiment of the invention showing a syringe assembled ready to use.

FIG. 3 is a schematic view of a syringe with the plunger retracted causing fluid intake.

FIG. 4 is a schematic view of a syringe after the plunger has been retracted and depressed, showing separation of the rear valve and front valve elements.

### DETAILED DESCRIPTION OF THE INVENTION

The apparatus (10) shown in the Figures comprises an elongate cylindrical barrel (12) having a first closed end (16) and a restricted second end (14) having a cone (18) for attaching a needle. The barrel may be graduated to facilitate the measurement of the liquid drawn into the barrel (12). The barrel (12) contains a piston (20) and associated plunger (22). As used herein, “forward” or “front” shall refer to the second end (14) of the syringe and “rearward” or “rear” shall refer to the first end (16).

The piston (20) comprises a rear valve (24) and a front valve (26) both of which form a liquid-tight seal with the interior of the barrel (12). The rear valve (24) permits drawing liquid into the barrel (12) when the plunger (22) is retracted and the front valve (26) permits expelling the liquid through the second restricted end (14) when the plunger (22) is compressed. However, the rear valve (24) may only be retracted once in the barrel (12). The restricted end (14) and closed end (16) prevent the removal of the plunger (22) and piston (20) apparatus effectively eliminating the possibility of mechanical manipulation of the apparatus (10) to facilitate re-use.

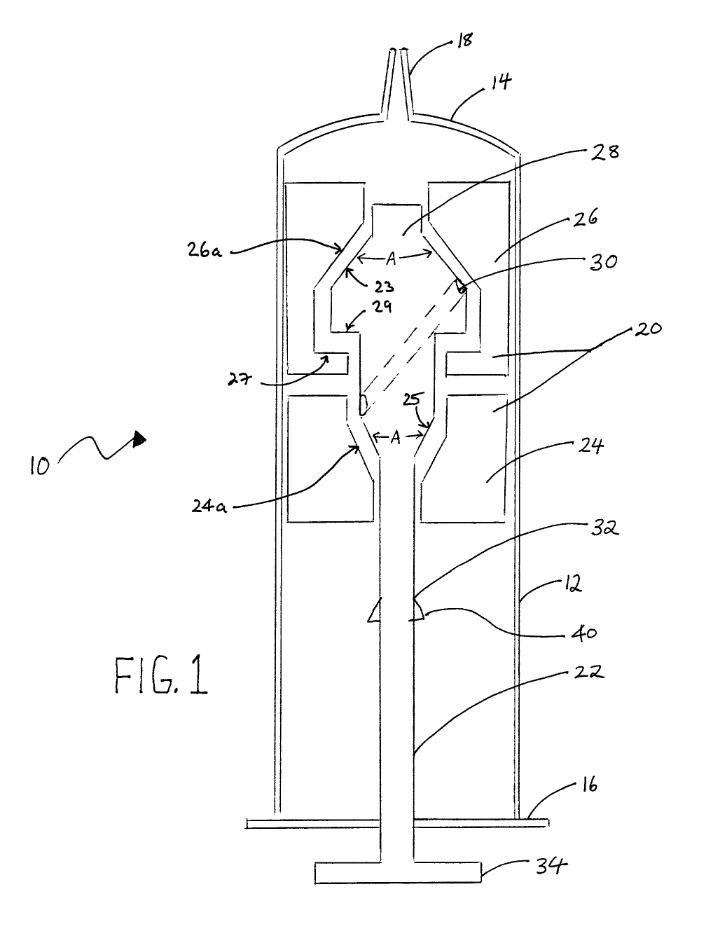

The plunger (22) and piston (20) are connected by means of an angular shaped plug (28) that is attached to the end of the plunger (22). The plug has a front sealing surface (23) and a rear sealing surface (25). As is shown in FIG. 1, the angularly shaped plug (28) couples with the reciprocally angled valve seats (24a, 26a) of the rear and front valves (24, 26) during liquid intake and expulsion. As depicted in FIG. 1, the plug has a bypass opening (30) passing through it to allow air flow following the expulsion of the liquid contents as will be described in greater detail later. The bypass opening (30) may be of any orientation. However, one end of the opening must be on the front sealing surface (23) or forward of the shoulder (27). The other opening must be rearward of the shoulder (27). The bypass opening (30) permits air to flow into the front portion of the barrel when the plunger and plug are retracted without the rear valve in contact with the plug. The bypass opening (30) may be a borehole through the plug (28) or it may take the form of a groove or grooves on the exterior surface of the plug (28).

The plunger (22) has a shaft (32) that is freely slidable within the barrel (12) and through the rear opening of the rear valve (24). The shaft (32) protrudes through and beyond the center of the closed end (16), ending in a handle (34) for moving the shaft (32).

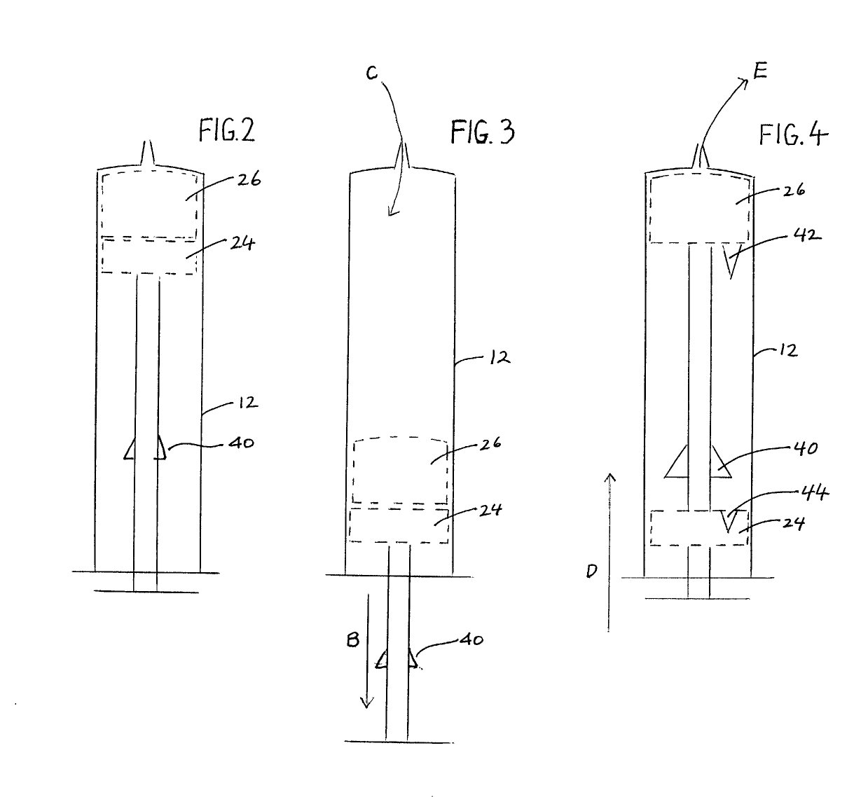

The operation of the invention will now be described having reference to the Figures. FIG. 2 represents the apparatus (10) ready for use. The plunger (22) is in a fully compressed position in the barrel (12) with the front and rear valves (26, 24) positioned together adjacent to the restricted end (14) of the barrel (12).

To draw liquid into the apparatus (10), the needle (not shown) attached to the cone (18) is immersed in the liquid and the plunger (22) is then retracted rearward. As the plunger (22) is retracted, the piston (20) moves up the barrel (12) as a single unit with the shoulder (29) of the angular plug (28) coupling with the front piston shoulder (27) and the rear sealing surface (25) engaging the rear valve seat (24a). As the plunger (22) is retracted, the plug (28) forms an air-tight seal with the rear valve (24) creating a low pressure area in the barrel (12) below the rear valve (24). The presence of this vacuum causes liquid to be drawn into the barrel (12) through the needle and cone (18) as is denoted by ‘C’ in FIG. 3. When the desired volume has been drawn the user stops retracting the plunger (22) leaving the plunger (22) and piston valves in the position shown in FIG. 3.

To expel the liquid, the plunger (22) is urged forward as is depicted in FIG. 4. As is evident from FIG. 1, upon compression, the plug (28) will disengage from the rear valve (24) but will engage the front valve (26) forming a tight seal between the front sealing surface (23) and the front valve seat (26a). The front valve (26) will form a liquid-tight seal with the interior of the barrel (12), expelling the contents of the barrel (12) through the cone (18). The rear valve (24) remains lodged in the position it was in prior to the compression of the plunger (22) as is evidenced in FIG. 4. With the valves in this configuration, the bypass (30) passing through the plug (28) makes it impossible to get a liquid-tight seal to draw more liquid into the barrel (12). If the plunger (22) is retracted, the bypass (30) allows air to flow through the front valve (26) into that portion of the barrel (12) below the front valve (26). A vacuum cannot be created and accordingly, liquid cannot be drawn into the apparatus (10). Hence, the apparatus (10) can only be used once. As mentioned earlier, the closed end (16) and restricted end (14) prevent the removal or manipulation of the plunger (22) or piston (20) to overcome this disabling feature.

In one embodiment, the shaft may also have at least one safety barb (40) as is shown in FIG. 4. The barb (40) may be molded onto the shaft. If the rear valve is made of a pliable material, the safety barb (40) allows the rear valve (24) to slide over the barb (40) in a direction towards the plunger handle (34) when the plunger (22) is urged forward. Once the rear valve (24) has moved into a position between the barb (40) and the handle (34), as shown in FIG. 4, it is not able to pass back over the barb (40), permanently separating the front (26) and rear (24) valves. This feature makes it more difficult to manipulate the rear valve (24) into an operational position adjacent to the front valve (24) as shown in FIG. 2.

In another embodiment, the front valve (26) may have a projection (42) that can mate with a reciprocal cavity (44) in the rear valve (24). The projection (42) may be a flexibly resilient piece that may be bent so that the projection (42) and cavity (44) are aligned prior to use. However, as soon as the plunger (22) is compressed and the valves become separated the projection (42) disengages the cavity (44) and returns to a position that is not aligned with the cavity (44). The unaligned projection (44) will then prevent the valves from being forced back into an operational state by maintaining a separation between the front valve (26) and the rear valve (24).

The syringe described herein may be assembled by first assembling the plunger (22) and plug (28) within the front valve (26). To facilitate assembly, the front valve (26) may be a two piece assembly which are glued or otherwise affixed together once the plug is inserted into the front valve (26) cavity. The rear valve (24) may simply be slid over the handle end of the plunger (22) prior to the handle (34) being attached to the plunger shaft (32). The barrel (12) may include a forward portion and a rear portion which are glued or welded together once the plunger, plug and front and rear valve assembly is placed within the rear portion of the barrel and the handle end of the plunger passed through an opening in the closed rear end (16) of the barrel. The forward portion of the barrel may then be placed over the front valve and attached to the rear portion. Once the assembly is complete, the inner components of the syringe may only be manipulated if the barrel is destroyed or compromised.

As will be apparent to those skilled in the art, various modifications, adaptations and variations of the foregoing specific disclosure can be made without departing from the scope of the invention claimed herein.

What is claimed is:

1. A single use syringe comprising:
(a) an elongate barrel having an interior surface, a rear end and a forward restricted end having an opening through which liquid can pass, said forward end having a cone for attaching a needle;
(b) a piston disposed within the barrel comprising a first forward valve and a separate second rear valve, said first valve and second valve each forming a substantially liquid-tight seal with the interior surface of the barrel, said first valve defining a cavity and a front opening and having a front valve seat and a rear shoulder, and said second valve defining a cavity and rear opening and having a rear valve seat;
(c) an elongate plunger comprising a shaft terminating with a plug, said plug having a front sealing surface, a shoulder, a rear sealing surface and defining a bypass having an opening through the front sealing surface and another opening rearward of the shoulder;
(d) said plug disposed within the first valve cavity and the second valve cavity with the shaft passing through the rear opening of the second valve, and said valve sized and shaped such that when the plug is pulled back, the front sealing surface disengages the front valve seat, the plug shoulder engages the piston cavity shoulder, the rear sealing surface engages the rear valve seat and the plug and first and second piston valves are pulled rearward within the barrel as one unit; and when said plug is urged in a forward direction, the forward sealing surface engages the forward valve seat and the first piston valve and plug disengage the second piston valve.

2. The syringe of claim 1 wherein said shaft comprises blocking means allowing passage of the shaft through the second piston valve in the forward direction but preventing passage of the shaft through the second piston valve in the rearward direction.

3. The syringe of claim 2 wherein said blocking means comprises a barb member having an inclined surface and a perpendicular surface.

4. The single use syringe of claim 1 wherein said front valve having a projection fitting into a reciprocal cavity in said rear valve wherein the projection and cavity are aligned during liquid intake but unaligned following liquid expulsion.

5. A single use syringe comprising:
(a) a barrel having a closed forward end and a closed rear end, said forward end defining a cone and an opening for attaching a needle;
(b) a piston assembly including a front valve and a rear valve;
(c) a plunger including a shaft which passes through the rear valve and a plug wherein said plug mates in a fluid-tight manner with the front valve and with the rear valve and defines an internal bypass opening operative when the front valve and rear valve are separated;
(d) means for coupling the plug to the front valve such that when the plug is retracted, the front valve also retracts; and
(e) wherein the plug does not engage the rear valve when urged forward.
